SEQUENCE LISTING (33 sequences)

SEQ ID NO: 1 - DNA, 666 bp, Dermatophagoides pteronyssinus
actaacgcctgcagtatcaatggaaatgctccagctgaaatcgatttgcgacaaatgcgaactgtcactcccattcgtatgcaaggaggctgtggttcatgttgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgccgacgaccaaatgcacaacgtttcggtatctcaaactattgccaaatttacccaccaaatgcaaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatatgttgtcattctc

SEQ ID NO: 2 - DNA, 387 bp, Dermatophagoides pteronyssinus
gatcaagtcgatgtcaaagattgtgccaatcatgaaatcaaaaaagttttggtaccaggatgccatggttcagaaccatgtatcattcatcgtggtaaaccattccaattggaagccgttttcgaagccaaccaaaacacaaaaacggctaaaattgaaatcaaagcctcaatcgatggtttagaagttgatgttcccggtatcgatccaaatgcatgccattacatgaaatgcccattggttaaaggacaacaatatgatattaaatatacatggaatgttccgaaaattgcaccaaaatctgaaaatgttgtcgtcactgttaaagttatgggtgatgatggtgttttggcctgtgctattgctactcatgctaaaatccgcgat

SEQ ID NO: 3 - DNA, 906 bp, Dermatophagoides pteronyssinus
cgtccatcatcgatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctgcagtatcaatggaaatgctccagctgaaatcgatttgcgacaaatgcgaactgtcactcccattcgtatgcaaggaggctgtggttcatgttgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgccgacgaccaaatgcacaacgtttcggtatctcaaactattgccaaatttacccaccaaatgcaaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatatgttgtcattctc

SEQ ID NO: 4 - protein, 129 aa, Dermatophagoides pteronyssinus
DQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 5 - protein, 222 aa, Dermatophagoides pteronyssinus
TNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 6 - protein, 320 aa, Dermatophagoides pteronyssinus
MKIVLAIASLLALSAVYARPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 7 - protein, 302 aa, Dermatophagoides pteronyssinus
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 8 - protein, 351 aa, Artificial
  Derp2-Derp1 fusion
DQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRDTNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 9 - protein, 431 aa, Artificial
  Derp2-ProDerp1 fusion
DQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRDRPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 10 - protein, 351 aa, Artificial
  Derp1-Derp2 fusion
TNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVILDQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 11 - protein, 431 aa, Artificial
  ProDerp1-Derp2
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVILDQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 12 - DNA, 1053 bp, Artificial
  Derp2-Derp1 DNA
gatcaagtcgatgtcaaagattgtgccaatcatgaaatcaaaaaagttttggtaccaggatgccatggttcagaaccatgtatcattcatcgtggtaaaccattccaattggaagccgttttcgaagccaaccaaaacacaaaaacggctaaaattgaaatcaaagcctcaatcgatggtttagaagttgatgttcccggtatcgatccaaatgcatgccattacatgaaatgcccattggttaaaggacaacaatatgatattaaatatacatggaatgttccgaaaattgcaccaaaatctgaaaatgttgtcgtcactgttaaagttatgggtgatgatggtgttttggcctgtgctattgctactcatgctaaaatccgcgatactaacgcctgcagtatcaatggaaatgctccagctgaaatcgatttgcgacaaatgcgaactgtcactcccattcgtatgcaaggaggctgtggttcatgttgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgccgacgaccaaatgcacaacgtttcggtatctcaaactattgccaaatttacccaccaaatgcaaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatatgttgtcattctc

SEQ ID NO: 13 - DNA, 1293 bp, Artificial
  Derp2-ProDerp1 DNA
gatcaagtcgatgtcaaagattgtgccaatcatgaaatcaaaaaagttttggtaccaggatgccatggttcagaaccatgtatcattcatcgtggtaaaccattccaattggaagccgttttcgaagccaaccaaaacacaaaaacggctaaaattgaaatcaaagcctcaatcgatggtttagaagttgatgttcccggtatcgatccaaatgcatgccattacatgaaatgcccattggttaaaggacaacaatatgatattaaatatacatggaatgttccgaaaattgcaccaaaatctgaaaatgttgtcgtcactgttaaagttatgggtgatgatggtgttttggcctgtgctattgctactcatgctaaaatccgcgatcgtccatcatcgatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctgcagtatcaatggaaatgctccagctgaaatcgatttgcgacaaatgcgaactgtcactcccattcgtatgcaaggaggctgtggttcatgttgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgccgacgaccaaatgcacaacgtttcggtatctcaaactattgccaaatttacccaccaaatgcaaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatatgttgtcattctc

SEQ ID NO: 14 - DNA, 1053 bp, Artificial
  Derp1-Derp2 DNA
actaacgcctgcagtatcaatggaaatgctccagctgaaatcgatttgcgacaaatgcgaactgtcactcccattcgtatgcaaggaggctgtggttcatgttgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgccgacgaccaaatgcacaacgtttcggtatctcaaactattgccaaatttacccaccaaatgcaaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatatgttgtcattctcgatcaagtcgatgtcaaagattgtgccaatcatgaaatcaaaaaagttttggtaccaggatgccatggttcagaaccatgtatcattcatcgtggtaaaccattccaattggaagccgttttcgaagccaaccaaaacacaaaaacggctaaaattgaaatcaaagcctcaatcgatggtttagaagttgatgttcccggtatcgatccaaatgcatgccattacatgaaatgcccattggttaaaggacaacaatatgatattaaatatacatggaatgttccgaaaattgcaccaaaatctgaaaatgttgtcgtcactgttaaagttatgggtgatgatggtgttttggcctgtgctattgctactcatgctaaaatccgcgat

SEQ ID NO: 15 - DNA, 1293 bp, Artificial
  ProDerp1-Derp2 DNA
cgtccatcatcgatcaaaacttttgaagaatacaaaaaagccttcaacaaaagttatgctaccttcgaagatgaagaagctgcccgtaaaaactttttggaatcagtaaaatatgttcaatcaaatggaggtgccatcaaccatttgtccgatttgtcgttggatgaattcaaaaaccgatttttgatgagtgcagaagcttttgaacacctcaaaactcaattcgatttgaatgctgaaactaacgcctgcagtatcaatggaaatgctccagctgaaatcgatttgcgacaaatgcgaactgtcactcccattcgtatgcaaggaggctgtggttcatgttgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgccgacgaccaaatgcacaacgtttcggtatctcaaactattgccaaatttacccaccaaatgcaaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatatgttgtcattctcgatcaagtcgatgtcaaagattgtgccaatcatgaaatcaaaaaagttttggtaccaggatgccatggttcagaaccatgtatcattcatcgtggtaaaccattccaattggaagccgttttcgaagccaaccaaaacacaaaaacggctaaaattgaaatcaaagcctcaatcgatggtttagaagttgatgttcccggtatcgatccaaatgcatgccattacatgaaatgcccattggttaaaggacaacaatatgatattaaatatacatggaatgttccgaaaattgcaccaaaatctgaaaatgttgtcgtcactgttaaagttatgggtgatgatggtgttttggcctgtgctattgctactcatgctaaaatccgcgat

SEQ ID NO: 16 - DNA, 36 bp, Artificial
  primer
ggttgctcttccaacgatcaagtcgatgtcaaagat

SEQ ID NO: 17 - DNA, 37 bp, Artificial
  primer
catgctaaaatccgcgatacttaacgcctgcagtatc

SEQ ID NO: 18 - DNA, 36 bp, Artificial
  primer
gatactgcaggcgttagtatcgcggattttagcatg

SEQ ID NO: 19 - DNA, 32 bp, Artificial
  primer
gcggccgctcagagaatgacaacatatggata

SEQ ID NO: 20 - DNA, 36 bp, Artificial
  primer
gggctcgagaaaagagatcaagtcgatgtcaaagat

SEQ ID NO: 21 - DNA, 33 bp, Artificial
  primer
ggcggccgctcagagaatgacaacatatggata

SEQ ID NO: 22 - DNA, 36 bp, Artificial
  primer
catgctaaaatccgcgatcgtccatcatcgatcaaa

SEQ ID NO: 23 - DNA, 36 bp, Artificial
  primer
tttgatcgatgatggacgatcgcggattttagcatg

SEQ ID NO: 24 - DNA, 33 bp, Artificial
  primer
gggctcgagaaaagaactaacgcctgcagtatc

SEQ ID NO: 25 - DNA, 36 bp, Artificial
  primer
ccatatgttgtcattctcgatcaagtcgatgtcaaa

SEQ ID NO: 26 - DNA, 36 bp, Artificial
  primer
tttgacatcgacttgatcgagaatgacaacatatgg

SEQ ID NO: 27 - DNA, 33 bp, Artificial
  primer
ggcggccgctcaatcgcggattttagcatgagt

SEQ ID NO: 28 - DNA, 36 bp, Artificial
  primer
gggctcgagaaaagacgtccatcatcgatcaaaact

SEQ ID NO: 29 - DNA, 41 bp, Artificial
  primer
atcaccatcaccacggtatgcaagatcaagtcgatgtcaaa

SEQ ID NO: 30 - DNA, 40 bp, Artificial
  primer
cttggttagttagttattagagaatgacaacatatggata

SEQ ID NO: 31 - DNA, 41 bp, Artificial
  primer
atcaccatcaccacggtatgcaaactaacgcctgcagtatc

SEQ ID NO: 32 - DNA, 40 bp, Artificial
  primer
cttggttagttagttattaatcgcggattttagcatgagt

SEQ ID NO: 33 - DNA, 41 bp, Artificial
  primer
atcaccatcaccacggtatgcaacgtccatcatcgatcaaa

(19) United States
(12) Patent Application Publication
(10) Pub. No.: US 2008/0274059 A1
(43) Pub. Date: Nov. 6, 2008

(54) MITE FUSION PROTEINS

(75) Inventors: Philippe MOINGEON, Antony (FR); Ingrid Bulder, Amsterdam (NL); Veronique Bordas, Antony (FR); Laetitia Bussieres, Versailles (FR)

Correspondence Address: STITES & HARBISON PLLC, 1199 NORTH FAIRFAX STREET, SUITE 900, ALEXANDRIA, VA, 22314, US

(73) Assignee: STALLERGENES S.A., Antony Cedex (FR)

(21) Appl. No.: 11/869,294

(22) Filed: Oct. 9, 2007

(30) Foreign Application Priority Data
Oct. 6, 2006 (EP) 06291561.6

(51) Int. Cl. A61K 39/35; C07K 14/00; C07K 14/435; C12N 1/19; A61K 49/00; C12N 15/12; A61P 37/08; G01N 33/53; C12N 1/21; C12N 5/10

(52) U.S. Cl. 424/9.81; 530/402; 536/23.4; 435/252.33; 435/254.23; 435/254.21; 435/365; 435/362; 424/192.1; 436/501

(57) ABSTRACT

The present invention relates to fusion proteins comprising a Group I allergen and a Group II allergen from the genus Dermatophagoides, isolated nucleic acid coding for them and their use for the manufacture of a medicament to prevent or treat a mite allergic reaction.

The invention relates to a fusion protein, an isolated nucleic acid coding for it and its use for the manufacture of a medicament to prevent or treat a mite allergic reaction.

The contribution of mites to allergy in humans was first documented in the 1920s when Cooke and Kern found that dust from vacuum cleaner bags caused positive skin reactions in asthma patients. Later, Voorhorst and colleagues were the first to implicate mites found in dust in the development of dust allergy. These investigators conducted a survey of the European and American continents and discovered they all had astigmatid mites in the genus Dermatophagoides. The concentration of mites found in survey samples from carpets and bedding varied from 1 to 500 mites per gram of dust. Mite extracts produced positive skin reactions similar to those observed by Cooke and Kern 43 years earlier. This finding led to the conclusion that Dermatophagoides were one of the principal sources of house dust allergens. Later studies confirmed Dermatophagoides pteronyssinus to be the most abundant mite species worldwide.

Dust mite allergies are often characterised by ‘perennial’ or ‘year-round’ hayfever or allergic rhinitis: frequent sneezing, a runny, stuffy, itchy nose and irritated eyes. Asthma and eczema can also be triggered by a dust mite allergy.

The allergens produced by the genus Dermatophagoides mites fall mainly into two immunologically important groups: group I (Der p 1 Der f 1) and II (Der p 2, Der f 2). Der p 1 and Der p 2 are the major European house dust mite (HDM) allergens from Dermatophagoides pteronyssinus. Der f 1 and Der f 2 are the major allergens from Dermatophagoides farinae. Group I allergens are approximately 25,000 Da glycoproteins found primarily in mite feces. The Der p 1 allergen occurs at 10 mg/mL (0.2 ng per fecal pellet) in mite feces and elutes rapidly from fecal pellets. Der p 1 is a cysteine protease secreted by mites during digestion and is released in fecal pellets that are 10 μm to 40 μm in size. Group I allergens are structurally homologous (among house dust mites, most particularly Dermatophagoides pteronyssinus and Dermatophagoides farinae species). Group II allergens, found in mite bodies, are approximately 15,000 Da proteins, and like group I allergens, share structural homologies. Cross-reactivity between species-specific allergens is seen within each group, but is not documented across groups.

Chapman M D et al. (J Immunol. 1980 August; 125(2):587-92) have described the purification of the Der p 1 protein from D. pteronyssinus in 1980.

Group I and II mite antigens are the most clinically relevant to asthma, atopic dermatitis, and allergic rhinitis since more than 80% HDM (house dust mite) allergic patients exhibit high seric IgE titers directed to these two allergens. Currently, at least twenty one different allergen groups have been identified for the European dust mite.

The therapeutic options fall in three major categories. The first opportunity is allergen avoidance or reduction of the exposure. It may be difficult or expensive for house dust mite allergens. The second and most widely used therapeutic option is the prescription of classical symptomatic drugs like anti-histamines and steroids. Symptomatic drugs are safe and efficient; however, they do not alter the natural cause of the disease. The third therapeutic alternative is specific allergy vaccination which, in most cases, reduces or alleviates the allergic symptoms caused by the allergen in question. Conventional specific allergy vaccination is a causal treatment for allergic disease. It interferes with basic immunological mechanisms resulting in persistent improvement of the patients' immune status. Thus, the protective effect of specific allergy vaccination extends beyond the treatment period in contrast to symptomatic drug treatment. Some patients receiving the treatment are cured, and, in addition, most patients experience a relief in disease severity and symptoms experienced, or at least an arrest in disease aggravation. Thus, specific allergy vaccination has preventive effects reducing the risk of rhinitis developing into asthma, and reducing the risk of developing new sensitivities.

The immunological mechanism underlying successful allergy vaccination is not known in detail. It is generally accepted that an active vaccine must have the capacity to stimulate allergen specific T-cells, preferably TH1 and regulatory T cells.

Specific allergy vaccination is, in spite of its virtues, not in widespread use, primarily for two reasons. One reason is the inconveniences associated with the traditional vaccination programme that comprises repeated vaccinations i.e. injections over several months. The other reason is, more importantly, the risk of allergic side reactions. Ordinary vaccinations against infectious agents are efficiently performed using a single or a few high dose immunizations. This strategy, however, cannot be used for allergy vaccination since a pathological immune response is already ongoing. Conventional specific allergy vaccination is therefore carried out using multiple subcutaneous or mucosal (e.g. sublingual) immunizations applied over an extended time period.

Recombinant allergens are an alternative to the complex biological extracts used in specific allergy vaccination with a better yield. Their biological properties as immunogenicity and harmlessness depend on the chosen expression system.

WO 88/10297 discloses the cDNA and amino acid sequences of the mature excreted Der p 1, the transient pre- and preproenzymes forms of Der p 1, and Der p 2. The nucleotide sequence of the cDNA insert of a phage clone reacted with the rabbit anti-Der p 1 serum results in the expression of a fusion protein comprising Der p 1 and a β-galactosidase. This recombinant chimera has been shown to inhibit IgE responses of mice to mature Der p 1.

Recombinant Der p 1 has been expressed in Pichia pastoris (Jacquet el al. Clin Exp Allergy. 2002 July; 32(7):1048-53.) as an hyperglycosylated protein with similar IgE binding and enzymatic activity but decreased histamine realising capacity as compared with the natural allergen.

Expression of Der p 2 in the baker's yeast yields a recombinant protein with properties similar to the naturel protein (Hakkaart G A, Harmsen M M, Chua K Y, Thomas W R, Aalberse R C, Van Ree R. Clin Exp Allergy. 1998 January; 28(1)45-52.).

Recombinant Der f 1 has been produced in insect cells, using a baculovirus expression system (Shoji H. et al., Biosci Biotechnol Biochem. 1996 April; 60(4):621-5; Shoji H. et al., Biosci Biotechnol Biochem. 1997 October ;61(10):1668-73) and in Aspergillus oryzae (Shoji H. et al., Biosci Biotechnol Biochem. 1999 April; 63(4):703-9).

Der f 2 has been expressed in E. coli (Iwamoto M. et al., Int Arch Allergy Immunol. 1996 April; 109(4):356-61).

The Applicant has cloned proDer p 1 in tobacco plants (WO2004/005334) and thus expressed four polypeptides recognized by a specific anti-Der p 1 antibody. Three of them are synthesis intermediate species of the mature Der p 1 protein and the fourth one has five amino acids more than the mature Der p 1 protein. It is also shown that the propeptide in proDer p 1 is essential for the expression of a recombinant Der p 1 in a eukaryotic cell (WO 2004/005334). Expression in various prokaryotic and eukaryotic systems (E. coli, yeast, CHO, Drosophila cells) have established that the propeptide is necessary for the expression of a well folded molecule: more specifically, expression of mature Der p 1 (i.e. without the propeptide) results in low levels of expression of a Der p 1 molecule which is incorrectly folded.

To replace biological extracts, there is a need for a vaccine product combining several recombinant house dust mite allergens, with a high purity, preferably with natural conformation, with a conserved antigenicity and immunogenicity (both in terms of IgE, IgG, and T lymphocyte recognition) as compared with the natural allergens, and which can be produced in prokaryotic and/or eukaryotic systems.

This is achieved by a fusion protein comprising a Group I allergen and a Group II allergen from the genus Dermatophagoides, the allergens having in the fusion protein a conformation close to the one of the natural mature allergens.

The present Inventors have expressed four fusion proteins assembling the two major allergens Der p 1 and Der p 2 from the common house dust mite Dermatophagoides pteronyssinus. Der p 2 has been fused at the N-or C-terminal end of either mature Der p 1 or proDer p 1. These four fusion proteins have been successfully expressed in Escherichia coli. The two forms with the Der p 1 propeptide have also been expressed in the yeast Pichia pastoris. These 6 fusion proteins are recognized in immunoblot analysis by anti-Der p 2 monoclonal antibodies and by IgE from house dust mite allergic patients. The inventors also observed some reactivity with a mouse monoclonal antibody and sheep polyclonal antibodies specific for native Der p 1. Immunoreactivity data confirm that both Der p 1 and Der p 2 are correctly folded in those fusion proteins since some conformational epitopes can be recognized by various antibodies directed to the distinct components of the fusion proteins.

Hence, these results support that a fusion protein can be successfully prepared by fusioning directly together at least two allergens while retaining the folding and immunogenicity of the native antigens.

### Fusion Protein

Thus, the present invention relates to a fusion protein comprising a Group I allergen and a Group II allergen from the genus Dermatophagoides.

As used herein, a “protein” or “peptide” denotes a molecule comprised of a linear array of amino acid residues connected to each other in the linear array by peptide bonds.

A “fusion protein” refers to a protein having at least a Group I allergen and a Group II allergen from the genus Dermatophagoides covalently linked, either directly or via a linker peptide. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order. Alternatively, the fusion protein could be obtained by chemical coupling.

A linker peptide sequence may be employed to separate the two polypeptide components by a distance sufficient to ensure that each polypeptide folds into its secondary and tertiary structures. This linker sequence may generally be from 1 to about 50 amino acids in length. Linker sequences are not required when the second polypeptide has a dispensable N-terminal region that can be used to separate the functional domains and prevent steric hindrance.

However, in the frame of the present invention, the Group I allergen and the Group II allergen of the fusion protein are preferably directly linked, in order to preclude any risk of immunological reaction to the linker itself.

Accordingly, the Group II allergen is preferably fused directly at the N- or C-terminal end of the Group I allergen. According to one embodiment of the present invention, the fusion protein consists in a N-or C-terminal fusion of a Group I allergen and group II allergen from the genus Dermatophagoides.

An “allergen” is defined as a substance, usually a peptide or protein, which elicits the production of IgE antibodies in predisposed individuals. Similar definitions are presented in the following references: Clin. Exp. Allergy, No. 26, pp. 494-516 (1996); Mol. Biol. of Allergy and Immunology, ed. R. Bush, Immunology and Allergy Clinics of North American Series (August 1996).

A “Group I allergen from the genus Dermatophagoides” is a peptide or protein retaining substantially the immunogenicity of a natural Group I allergen from the genus Dermatophagoides. Preferably, the Group I allergen is Der p 1 or Der f 1, or a proform thereof.

A “Group II allergen from the genus Dermatophagoides” is a protein retaining substantially the immunogenicity of a natural Group II allergen from the genus Dermatophagoides. Preferably, the Group II allergen is Der p 2 or Der f 2, or a proform thereof.

A “proform” means a precursor of the mature protein, the mature protein being generally obtained by enzymatic processing of the proform. A proform includes preproteins (with a leader peptide), proproteins (with a propeptide) and preproproteins, for example proDer p 1 and preproDer p 1.

“Retaining substantially the immunogenicity of a natural allergen” means retaining completely or partially the ability to induce and react with IgE and IgG antibodies directed towards said natural allergen. It includes in particular allergens with reduced IgE reactivity (hypoallergenic forms).

Consequently, a “Group I (or II) allergen from the genus Dermatophagoides” includes peptides or proteins which comprise or consist of the sequence of a natural Group I (or II) allergen, variants, mutants, fragments, especially those encoding selected B and/or T epitopes, as well as homologs thereof with a percentage of identity above 80%, preferably 90%, still preferably 95%, provided that they substantially retain the immunogenicity of a natural Group I (or II) allergen from the genus Dermatophagoides.

In one embodiment according to the invention, the Group II allergen is Der p 2 (SEQ ID NO:4) and the Group I allergen is Der p 1 (SEQ ID NO:5) or proDer p 1 (SEQ ID NO:7). Preferably, the fusion protein according to the invention comprises or consists in SEQ ID NO:8 (Der p 2-Der p 1 fusion), SEQ ID NO:9 (Der p 2-ProDer p 1 fusion), SEQ ID NO:10 (Der p 1-Der p 2 fusion), SEQ ID NO:11 (ProDer p 1-Der p 2 fusion). These four fusion proteins are here referred as F1, F2, F3 and F4 respectively.

In another embodiment, the Group II allergen consists in Der f 2 and the Group I allergen is Der f 1 or proDer f 1.

As used herein, a variant sequence is a naturally occurring sequence but which diverges from the reference sequence by some point mutations.

As used herein, a mutant sequence is a sequence of a natural allergen in which one or several mutations have been introduced, for example to abrogate N-glycosylation sites, increase or decrease the cysteine protease activity of said allergen, improve the expression of the protein in an appropriate expression system, for example in E. coli, notably taking into account the codon bias, or improve T-cell epitopes anchoring sites so as to enhance the association with MHC class I or class II. Preferred point mutations of the Der p 1 allergen are mutations of the N-glycosylation sites (N34Q and/or N150Q in SEQ ID NO:6) or of the catalytic site (C132V in SEQ ID NO:6).

A fusion protein according to the invention may further comprise at least one additional allergen from the genus Dermatophagoides. For example, it may comprise a Group V and/or a Group VII and/or a Group XIII allergen from the genus Dermatophagoides, preferably Der p 5, Der p 7, Der p 13 from Dermatophagoides pteronyssinus or Der f 5, Der f 7, Der f 13 from Dermatophagoides farinae.

A fusion protein according to the invention may also comprise a molecule targeting or activating immune cells. Examples of such molecules include immunoglobulin Fc fragments, the B subunit of cholera toxin or integrins ligands (e.g. Mascarell et al., Vaccine. Apr. 24, 2006; 24(17):3490-9); Sun et al., Scand J Immunol. 2006 September; 64(3):251-9).

In other embodiments, the fusion protein could contain, in amino or carboxyterminal position, a tag (for instance a histidine tag), or another moiety which may increase expression, solubility, stability of the molecule, affect the secretion, maturation, post-translational modifications, or facilitate addressing to different subcellular compartments (endoplasmic reticulum, golgi apparatus, endosomes, periplasm . . . ). This additional fragment can be fused so that it can be cleaved off.

### Nucleic Acids and Methods of Expression

The isolated nucleic acids, also named polynucleotides, such as DNA or RNA molecules, that encode the fusion proteins defined above are also part of the invention, while taking into account the degeneracy of the genetic code. They can be obtained by standard techniques well known by the one skilled in the art, such as in vitro DNA amplification or polymerisation, in vitro gene synthesis, oligonucleotides ligation, or by a combination of these techniques.

Accordingly, the present invention provides a nucleic acid which comprises a sequence coding for a fusion protein according to the invention.

The proteins or nucleic acids of the invention are advantageously in isolated or purified form.

By “purified” and “isolated” it is meant, when referring to a protein or a nucleotide sequence, that the indicated molecule is present in the substantial absence of other biological macromolecules of the same type. The terms “purified” and “isolated” as used herein preferably mean at least 75% by weight, more preferably at least 85% by weight, more preferably still at least 95% by weight, and most preferably at least 98% by weight, of the protein or nucleotide sequence of interest. An “isolated” or “purified” nucleic acid molecule which encodes a particular polypeptide refers to a nucleic acid molecule which is substantially free of other nucleic acid molecules that do not encode the subject polypeptide. However, the molecule may include some additional bases or moieties which do not deleteriously affect the basic characteristics of the composition.

Briefly, the sequences encoding the polypeptide components of the fusion protein may be assembled separately, and ligated into an appropriate expression vector. The 3′ end of the DNA sequence encoding one polypeptide component is ligated, with or without a linker sequence, to the 5′ end of a DNA sequence encoding the second polypeptide component so that the reading frames of the sequences are in phase. This permits translation into a single fusion protein that retains the immunogenicity of both component polypeptides.

The DNA fragments encoding the polypeptide components of the fusion protein can also been excised and used as DNA templates for a overlap-PCR with suitable primer pairs. Particularly, the present invention provides a nucleic acid which comprises, consists essentially of, or consists of a fusion of the two sequences SEQ ID NO:1 (mature Der p 1) and SEQ ID NO:2 (mature Der p 2), or of SEQ ID NO:3 (proDer p 1) and SEQ ID NO:2.

As will be understood by those of skill in the art, it may be advantageous in some instances to produce fusion protein-encoding nucleotide molecules possessing codons non-naturally occurring in the components of the fusion. For example, codons preferred by a particular prokaryotic or eukaryotic host can be selected to increase the rate of recombinant protein expression or to produce a recombinant RNA transcript having desirable properties, such as a longer half-life. It also may be advantageous, for example using site-directed mutagenesis, to alter N-glycosylation sites, enzymatic active sites, B or T-cells epitopes, including IgE binding epitopes.

A nucleic acid according to this invention can also include sequences encoding tags, carriers proteins, signal peptides, or non transcribed or translated sequences increasing expression or stability of the molecule.

The invention also provides nucleic acid sequences that are hybridizable to any of the above sequences or their complementary sequences under standard hybridization conditions, preferably conditions of high stringency.

A nucleic acid molecule is “hybridizable” to another nucleic acid molecule, when a single stranded form of the nucleic acid molecule can anneal to the other nucleic acid molecule under the appropriate conditions of temperature and solution ionic strength (see Sambrook et al., 1989). The conditions of temperature and ionic strength determine the “stringency” of the hybridization. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. For hybrids of greater than 100 nucleotides in length, equations for calculating Tm have been derived (see Sambrook et al., supra, 9.50-9.51). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tm of 55° C., and utilizes conditions as set forth above. In a preferred embodiment, the Tm is 60° C. or even preferred 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

The present invention further relates to cloning and/or expression vectors comprising a nucleic acid sequence of the invention, and to host cells comprising the nucleic acid of the invention or said vector, i.e. a host cell wherein at least one of these nucleic acids or vectors was transferred. The expression vector according to the invention may comprise a functional expression cassette which is also an object of the present invention. An expression cassette comprises a nucleic acid sequence encoding a fusion protein of the invention, which is operably linked to elements necessary to its expression. Said vector advantageously contains a promoter sequence, signals for initiation and termination of translation, as well as appropriate regions for regulation of translation. Its insertion into the host cell may be transient or stable. Said vector may also contain sequences encoding specific signals which trigger the secretion of the translated protein or its targeting to cellular compartments or organelles (e.g; Golgi apparatus, endosomes, periplasm . . . ).

These various control signals are selected according to the host cell and may be inserted into vectors which self-replicate in the host cell, or into vectors which integrate the genome of said host.

Host cells may be prokaryotic or eukaryotic, including but not limited to bacteria, yeasts, plant cells, insect cells, mammalian cells, including cell lines which are commercially available. Preferred examples for expression hosts are Escherichia coli, Lactobacilli, probiotic bacteria, Pichia pastoris, Saccharomyces cerevisiae, insect cells, plant cells, COS cells and CHO cells.

The transfection of the host cell may be performed using any standard technique, such as chemical transformation, electroporation, phosphate calcium precipitation or lipofection.

Alternatively, the fusion protein according to the invention is expressed in vitro with a cell-free transcription and translation system from a DNA or RNA matrix containing required elements for its expression in a cell lysate or reconstituted system (for example, Rapid Translation System®, Roche Diagnostics or Retic Lysate IVT™, Ambion).

The recombinant protein can then be recovered and purified, by means of well-known procedures for purification: it may be purified from lysates or cell extracts, inclusion bodies or from the culture supernatant by methods such as HPLC chromatography, immunoaffinity techniques with specific antibodies, and the like.

### Vaccinal Compositions, Medicaments and Methods for Preventing or Treating an Allergic Reaction Against Mite in an Individual

The present invention also relates to the use of a fusion protein as defined above, for the manufacture of a medicament intended for preventing and/or treating a mite allergic reaction.

The present invention also relates to an immune or vaccinal composition comprising a fusion protein according to the invention, in a pharmaceutically acceptable carrier.

The present invention also relates to a method for preventing or treating an allergic reaction comprising administering an individual in need thereof with a prophylactically or therapeutically effective quantity of a fusion protein as defined above.

As used herein, the term “immune composition” denotes a composition which is liable to induce an immune response when administered in an individual.

As intended herein, the term “vaccinal” relates to the capacity of a substance to prevent or to treat a pathological reaction of the immune system.

In the context of the invention, the terms “to treat”, “treating” or “treatment”, means reversing, alleviating, or inhibiting the course of a pathological reaction of the immune system or one or more symptoms thereof.

In the context of the invention, the terms “to prevent” or “preventing”, means the onset of a pathological reaction of the immune system or one or more symptoms thereof.

As used herein, the term “individual” preferably denotes a human, but may more generally a mammal, such as a rodent, a feline, a canine, and a primate.

The suitable immune or vaccinal compositions may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

“Pharmaceutically” or “pharmaceutically acceptable” refers to molecular entities and compositions that do not produce adverse, allergic or other untoward reactions when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Its use in the vaccinal compositions, in the medicaments, or for implementing the methods for preventing or treating a pathological reaction of the immune system in an individual according to the invention is contemplated.

In the frame of methods for preventing or treating allergic reactions, the vaccinal compositions or the medicaments, according to the invention, can include any conventional vaccination adjuvant, including heat-labile enterotoxin (LT), cholera-toxin (CT), cholera toxin B subunit (CTB), polymerised liposomes, mutant toxins.

For oromucosal administration, the adjuvants may preferably be a Bifidobacterium, a lactic acid bacterium (either in the form of a cell suspension, freeze-dried cells, a lysate, purified sub-components, or purified molecules), or a combination of a corticosteroid with vitamin D3 or any metabolite or analog of the latter.

Advantageously, where mucosal administration is contemplated, the adjuvant may be a synthetic particulate vector that comprises a non-liquid hydrophilic core which comprises a cross-linked polysaccharide. Accordingly, the fusion protein according to the invention may be formulated in a mucoadhesive formulation based on a synthetic particulate vector that comprises (i) a particle comprising a non-liquid hydrophilic core which comprises a cross-linked polysaccharide; and (ii) a fusion protein according to the invention. Such a formulation was found to be particularly efficient in inducing immune tolerance The particles which can be used are described in the international patent application PCT/IB2007/002379.

Briefly, the cross-linked polysaccharide may be derived from any saccharide monomers, preferably glucose. The polysaccharides preferably have a molecular weight between 2,000 to 100,000 daltons, and most preferably 3,000 to 10,000 daltons. Preferred polysaccharides are starch (glucose alpha 1-4 polymers) and dextran (glucose alpha 1-6 polymers derived from bacteria), or hydrolysates thereof such as dextrins or maltodextrins.

Ionic groups, i.e. anionic (e.g. sulfate or carboxylate) or cationic groups (e.g. quaternary ammonium ions, and primary, secondary, or tertiary amines) are optionally grafted to the core of cross-linked polysaccharide (preferably 0 to 3 milliequivalents, more preferably 0 to 2 milliequivalents, of ionic charge per gram).

Optionally, the cross-linked polysaccharide core is at least partially coated with a layer of amphiphilic compounds andlor a layer of lipidic compounds.

The diameter of the particle may be comprised between 10 nm and 5 μm and preferably between 20 and 200 nm.

For parenteral administration in an aqueous solution, for example, the solution should be suitably buffered if necessary and the liquid diluent first rendered isotonic with sufficient saline or glucose. These particular aqueous solutions are especially suitable for intramuscular and subcutaneous administration. In this connection, sterile aqueous media which can be employed will be known to those of skill in the art in light of the present disclosure.

Preferably, the vaccinal composition, or the medicament is to be administered by the mucosal route, more preferably by the oromucosal route, and most preferably by the sublingual route. As such the vaccinal composition and the medicament are preferably formulated in a way adapted for such administration routes.

Mucosal administration denotes any administration method, wherein the formulation in part or in full comes into contact with a mucosa. Mucosa refers to the epithelial tissue that lines the internal cavities of the body. The mucosal surface may be selected from the group consisting of a nasal, buccal, oral, vaginal, ocular, auditory, pulmonary tract, urethral, digestive tract, and rectal surface.

Oromucosal administration comprises any administration method, wherein the formulation in part or in full comes into contact with the mucosa of the oral cavity and/or the pharynx of the patient. It includes in particular sublingual, perlingual (i.e. through the tongue mucosa) and oral administrations.

In the frame of methods for preventing or treating allergic reactions, the vaccinal compositions or the medicaments, according to the invention, the administration regimen may be maintained for instance for a period of less than 6 weeks to more than 3 years.

Preferably, in the frame of methods for preventing or treating allergic reactions, the range for the vaccinal compositions or the medicaments, according to the invention, may be between 0.001 mg/kg to 5 mg/kg/week. More preferably, the dose range is between 0.01 mg to 1 mg/kg and more preferably 0.03 mg/kg/week to 0.1 mg/kg/week. In preferred embodiments, the dose for the vaccinal compositions or the medicaments according to the invention is 0.056 mg/kg body weight once per week. It is further contemplated that the dose may be between 0.05 mg/kg to 3 mg/kg/week. Some variation in dosage will necessarily occur depending on the condition of the subject being treated. The person responsible for administration will, in any event, determine the appropriate dose for the individual subject.

### Detection/Diagnosis Applications

The fusion protein according to the invention may further be used to detect antibodies directed against a Group I allergen and/or a Group II allergen from the genus Dermatophagoides, in a sample from an individual.

Accordingly, the invention further provides a method for in vitro detecting antibodies directed against a Group I allergen and/or Group II allergen, in a biological sample from an individual, which method comprises the steps consisting of:
a) contacting said biological sample from a patient with a fusion protein comprising a Group I allergen and a Group II allergen from the genus Dermatophagoides, according to the invention;
b) detecting formation of immune complexes between said fusion protein and antibodies in the biological sample;

whereby, if immune complexes are detected, then the biological sample contains antibodies directed against a Group I allergen and/or a Group II allergen from the genus Dermatophagoides.

The individual may be a human or a non-human animal, in particular a non-human mammal, such as a rodent, a feline, a canine, and a primate.

The biological sample may be in particular a biological fluid, such as blood or serum.

Detection of antibodies in the biological sample from an individual may indicate that said individual is sensitised, or allergic, to said Group I allergen and/or a Group II allergen from the genus Dermatophagoides.

The antibody may be an IgM, IgE, IgG or IgA antibody.

The skilled person may use any appropriate qualitative or quantitative method known in the art, to detect the antibodies. The assay may be carried out by immobilising the fusion protein on a solid phase, or conversely with the fusion protein is the fluid phase. Typical methods which may be used include ELISA, Western blotting.

Where the concentration of the antibodies is determined, quantitation of the antibody response may be repeated in time, for instance in order to monitor efficacy of a desensitization treatment administered to the individual.

The fusion protein according to the invention may further be used for cellular tests such as a T-cell proliferation test, mediator release test etc. The fusion protein may be exposed to various types of cells in order to elicit measurable responses. Such responses may comprise the release of histamine or other mediators (e.g., leukotriens, serotonine, ECP) in the case of allergic effector cells (e.g., basophils mast cells, eosinophils). In another type of assay the proliferation or death (e.g., apoptosis) of cells may be measured e.g., by the uptake of .sup.3H Thymidine or any other suitable assay. Such cells may be T cells. Furthermore, fusion proteins may be used to induce the release of cytokines or other immunologically relevant substances (e.g., from T cells) that can be measured. Such cellular tests can be performed for instance on PBMC collected from an individual.

Since fusion proteins can contain epitopes of unrelated allergens they may be used for diagnostic screening tests (in vitro, in vivo as outlined above) in order to detect sensitization or unresponsiveness of an individual against one of the components of the fusion protein. This may allow providing the physician with a diagnostic test which is suited to screen for sensitized patients in a fast way.

Thus the fusion protein according to the invention may also be used for diagnostic purposes, for instance for in vivo provocation testing. Such tests may comprise skin testing (e.g., skin prick or intradermal testing), nasal provocation testing, all forms of food challenge testing or bronchial provocation testing.

In particular, a method of diagnosis according to the invention may comprise the steps of (a) injecting by intradermal or subcutaneous route the fusion protein according to the invention; and (b) detecting IgE reactivity, in particular by measuring the diameter of the wheal and flare reaction at the site of injection, wherein an IgE reactivity is indicative of an individual sensitised or allergic to a Group I and/or Group II allergen from the genus Dermatophagoides.

The invention further provides a method of detecting an IgE reactivity in an individual which comprises the steps of (a) injecting by intradermal or subcutaneous route the fusion protein according to the invention; and (b) detecting a wheal and flare reaction at the site of injection, wherein a wheal and flare reaction is indicative of an IgE reactivity of the individual to a Group I and/or Group II allergen from the genus Dermatophagoides.

According to an embodiment the diameter of the wheal and flare reaction at the site of injection may be measured to quantify the IgE reactivity.

The invention also relates to the use of a fusion protein comprising a Group I allergen and a Group II allergen from the genus Dermatophagoides, according to the invention, for the manufacture of a diagnostic test. Said diagnostic test further makes part of the invention and is intended to be used for screening of patients sensitized to Group I and/or Group II Dermatophagoides allergens.

The invention will be further illustrated in view of the following figures and examples.

### FIGURES

FIG. 1: Schematic representation of the Der p 1-Der p 2 fusion proteins

FIG. 2: Production of the Mite Fusion Proteins in Pichia pastoris

Western blot analysis of culture supernatants after 15 h induction in BMMY medium with specific monoclonal antibodies against the Der p 2 (A) and the Der p 1 (B) moities of the fusion proteins. (A): negative control strain (transformed with pPICZα empty vector; lane 1), F1 (lane 2), F2 (lane 3), F3 (lane 4), F4 (lane 5), recombinant proDer p 1 expressed in tobacco (lane 6, patent WO2004005334), recombinant Der p 2 (lane 7). (B): negative control strain (lane 1), F2 (lane 2), F4 (lane 3), recombinant proDer p 1 (lane 4), recombinant Der p 2 (lane 5). Molecular weights (kDa) are indicated on the left.

FIG. 3: IgE Reactivity of the Recombinant Mite Fusion Proteins Produced in Yeast

Immunoblot analysis of culture supernatants for F2 (lane 2) and F4 (lane 3) with a pool of sera from house dust mite allergic patients. Controls are recombinant Der p 1 (lane 4) and recombinant Der p 2 (lane 5). Molecular weights (kDa) are indicated on the left.

FIG. 4: Deglycosylation of Mite Fusion Proteins Expressed in Yeast

Buffer exchange of culture supernatant was performed by ultrafiltration. Proteins were treated with endoglycosidase H prior to SDS-PAGE analysis. Non treated (lane 1) and treated (lane 2) negative control supernatant, non treated (lane 3) and treated (lane 4) F2 protein, non treated (lane 5) and treated (lane 6) F4 protein. Lane 7: recombinant Der p 2. Numbers on the left indicate molecular weights (kDa).

FIG. 5: Production of Mite Fusion Proteins in Escherichia coli

Der p 2 (A) and IgE (B) reactivities of in vitro expressed mite fusion proteins. F1 (lane 1), F2 (lane 2), F3 (lane 3), F4 (lane 4), recombinant Der p 2 (lane 5, yeast culture supernatant). (C) anti Der p 2 western blot analysis of mite fusion protein expressed in vivo: negative control (1), F1 (2), F2 (3), F3 (4), F4 (5), recombinant Der p 1 (6), recombinant Der p 2 (8). Molecular weights (kDa) are indicated on the left sides of the images.

### EXAMPLE

### Materials and Methods

Construction of Mite Fusion Proteins:

For Expression in Pichia pastoris

The XhoI-NotI fragments from pPICZα-Der p 1 and pPICZα-Der p 2 (Sanquin, Netherlands) were used to generate the F1 fusion (Der p 2- Der p 1) by an overlap-PCR using the following oligonucleotides: Dp2FW (SEQ ID NO:16: GGTTGCTCTTCCAACGATCAAGTCGATGTCAAAGAT), Dp2MDp1FW (SEQ ID NO:17: CATGCTAAAATCCGCGATACTTAACGCCTGCAGTATC), Dp2MDp1RV (SEQ ID NO:18: GATACTGCAGGCGTTAGTATCGCGGATTTTAGCATG), Dp1-RV (SEQ ID NO:19: GCGGCCGCTCAGAGMTGACAACATATGGATA). The PCR fragment was then cloned into pGEM-T vector (Promega). This plasmid subsequently used as a template for PCR with the Dp2_FW_XhoI (SEQ ID NO:20: GGGCTCGAGAAAAGAGATCAAGTCGATGTCAAAGAT) Dp1_RV_NotI (SEQ ID NO:21: GGCGGCCGCTCAGAGAATGACMCATATGGATA) primers. The amplified fragment was cloned in frame with the alpha factor propeptide sequence into pPICZα vector (Invitrogen) at the XhoI and NotI sites. The F2 (Der p 2-proDer p 1), F3 (Der p 1-Der p 2) and F4 (proDer p 1-Der p 2) chimeras were directly cloned into pPICZα plasmid at the XhoI and NotI sites after an amplification by overlap-PCR using the XhoI-NotI fragments from pPICZα-Der p 1 and pPICZα-Der p 2 (Sanquin, Netherlands) as DNA templates. The primers we used were: Dp2_FW_XhoI, Dp2PDp1FW (SEQ ID NO:22: CATGCTAAAATCCGCGATCGTCCATCATCGATCAAA), Dp2PDp1RV (SEQ ID NO:23: TTTGATCGATGATGGACGATCGCGGATTTTAGCATG) and Dp1_RV_NotI for F2 fusion, Dp1_FW_XhoI (SEQ ID NO:24: GGGCTCGAGAAAAGMCTAACGCCTGCAGTATC), Dp1Dp2FW (SEQ ID NO:25: CCATATGTTGTCATTCTCGATCAAGTCGATGTCAAA), Dp1Dp2RV (SEQ ID NO:26: TTTGACATC-GACTTGATCGAGAATGACMCATATGG), Dp2_RV_NotI (SEQ ID NO:27: GGCGGCCGCTC-AATCGCGGATTTTAGCATGAGT), for F3 and PDp1_FW_XhoI (SEQ ID NO:28 GGGCTC-GAGAAAAGACGTCCATCATCGATCAAAACT), Dp1Dp2FW (SEQ ID NO:25: CCATATGTTGTCAT-TCTCGATCAAGTCGATGTCAAA) Dp1Dp2RV (SEQ ID NO:26: TTTGACATCGACTTGATCGAGMT-GACAACATATGG), and Dp2_RV_NotI.

For Expression in E. coli

The different constructs were cloned into pIX2.0 according to the manufacturer instructions (Qiagen). pPICZα derived plasmids described above served as PCR template. The gene-specific primer pairs were His_Dp2_FW (SEQ ID NO:29: ATCACCATCACCACGGTATGCAAGAT-CAAGTCGATGTCAAA)/Dp1_RV (SEQ ID NO:30: CTTGGTTAGTTAGTTATTAGAGAATGACAAC-ATATGGATA) for fusions F1 and F2; His_Dp1 (SEQ ID NO:31 : ATCACCATCACCACGGTAT-GCAAACTAACGCCTGCAGTATC)/Dp2_RV (SEQ ID NO:32: CTTGGTTAGTTAGTTATTAATCG-CGGATTTTAGCATGAGT) for fusion F3 and His_PDp1_FW (SEQ ID NO:33 ATCACCATCA-CCACGGTATGCAACGTCCATCATCGATCAAA)/Dp2_RV for fusion F4.

In vitro Expression in E. coli Lysate

Proteins were expressed in E. coli lysate using the EasyXpress Maxi kit (Qiagen) with 10 μg of plasmid DNA template according to the manufacturer instructions and protein contents were analysed by western blot.

In vivo Expression in E. coli

The fusion proteins were expressed in E. coli BL21 (DE3) pLysS strain. Cells were transformed with the different pIX2.0 vectors by the heat shock method according to the provider instructions (Invitrogen). Transformed clones were selected on LB plates containing Carbenicillin (50 μg/ml) and Chloramphenicol (20 μg/ml). The construct integrity was checked by PCR.
Single colonies were inoculated in LB Cb (50 μg/ml) Cam (20 μg/ml) medium and grown overnight at 37° C. Overnight cultures were diluted 1:100 in LB Cb (50 μg/ml) and grown until the OD₆₀₀ₙₘ was between 0.4 and 0.8. Target protein expression was then induced with 1 mM IPTG (Sigma Aldrich). Cell pellets were collected by centrifugation and stored at −20° C. for further analysis. Lysis was performed according to the following procedure. Cell pellets were first frozen in liquid nitrogen and thawed at 37° C. five times, resuspended in lysis buffer (50 mM Tris pH8 50 mM NaCl 1 mM EDTA) and incubated on a tube rotator for 30 minutes at 4° C. Samples were then sonicated (10 times 10 seconds, 50W, on ice) and clarified by centrifugation. Lysis supernatants were submitted to western blot analysis.

Expression in Yeast

The fusion proteins were expressed in methanotrophic P. pastoris GS115 or X33 strains. Transformation was performed according to the manufacturer instructions (Invitrogen). Briefly, pPICZα plasmids containing the different constructs were digested with SacI and digested plasmids were used to transform yeast cells by electroporation. Positive clones were selected on YPD zeocine (100 μg/ml) plates and integration of the constructs was checked by PCR.

Selected clones were inoculated in buffered glycerol complex medium (BMGY pH6) and grown overnight at 30° C. to an OD₆₀₀ₙₘ of 2-6. Cells were then diluted in buffered methanol complex medium (BMMY pH6) at an OD₆₀₀ₙₘ of 1 and growth was continued for 15 hours at 30° C. Culture supernatant was harvested by centrifugation and stored at −20° C. until use.

SDS-PAGE and Western Blot Analysis

Proteins were separated by SDS-PAGE using Nupage 4-12% Bis-Tris acrylamide gels with MES buffer under non-reducing conditions and then transferred onto nitrocellulose membranes as described by the manufacturer (Invitrogen). The molecular size marker is SeeBlue Plus2 (Invitrogen). Unreacted sites were blocked by incubating membranes in TBS buffer containing 1% non fat milk (BioRad). Reactivities of the Der p 2 and Der p 1 moities were then analysed with an anti Dpx antibody (0.2 μg/ml; Indoor Biotechnologies, USA), an in-house mouse monoclonal anti Der p 1 antibody (0.25 μg/ml) or serum pool from allergic patients (1:30). Incubations were done in TBS buffer supplemented with 0.2% non fat milk and 0.1% Tween 20. The secondary antibodies were an HRP conjugated sheep anti mouse IgG antibody (Sigma), or a rabbit anti human IgE antibody (Dakota) followed by an HRP conjugated goat anti rabbit IgG antibody (Calbiochem). For detection, we used either the West Pico or West Femto chemiluminescent substrates (Molecular Probes).

Endoglycosidase H Treatment

Proteins were placed in a 50 mM Sodium Acetate pH5.2 buffer by buffer exchange with Vivaspin 500 filters (PES MWCO 10 kDa, Sartorius). 0.04% SDS was subsequently added. 50 μl samples were incubated with or without 5 mU of endoglycosidase H (Roche Diagnostics) at 37° C. for 4 hours. Proteins were then analysed by SDS-PAGE.

### Abbreviations:

Cb: carbenicillin, Cam: chloramphenicol

### RESULTS

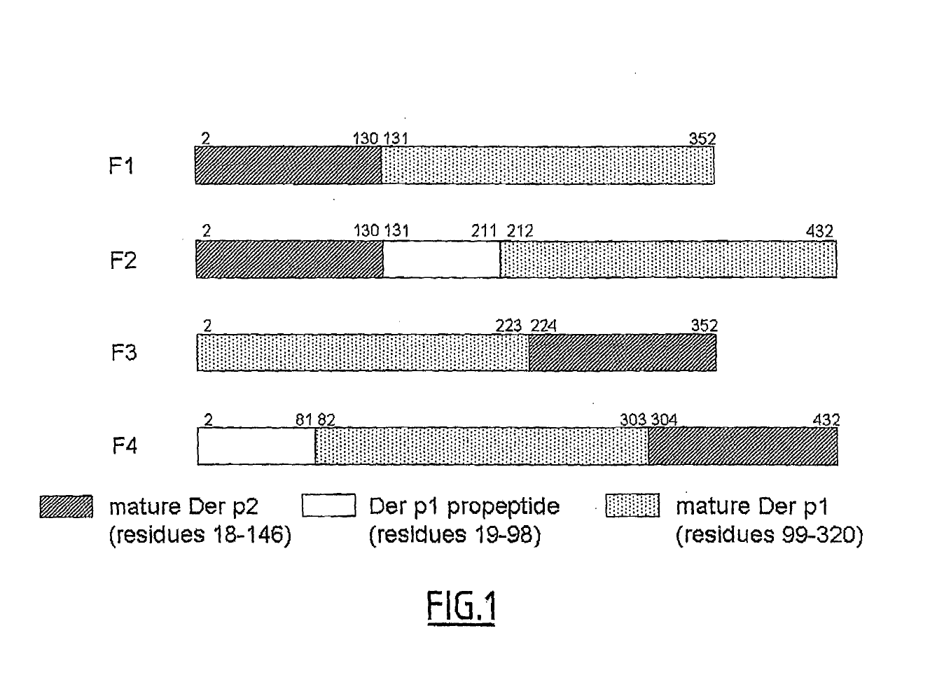

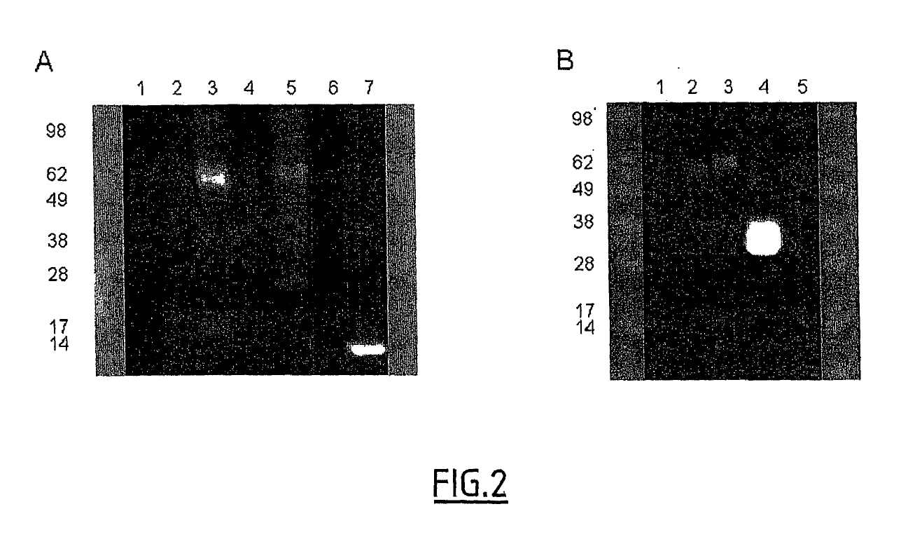

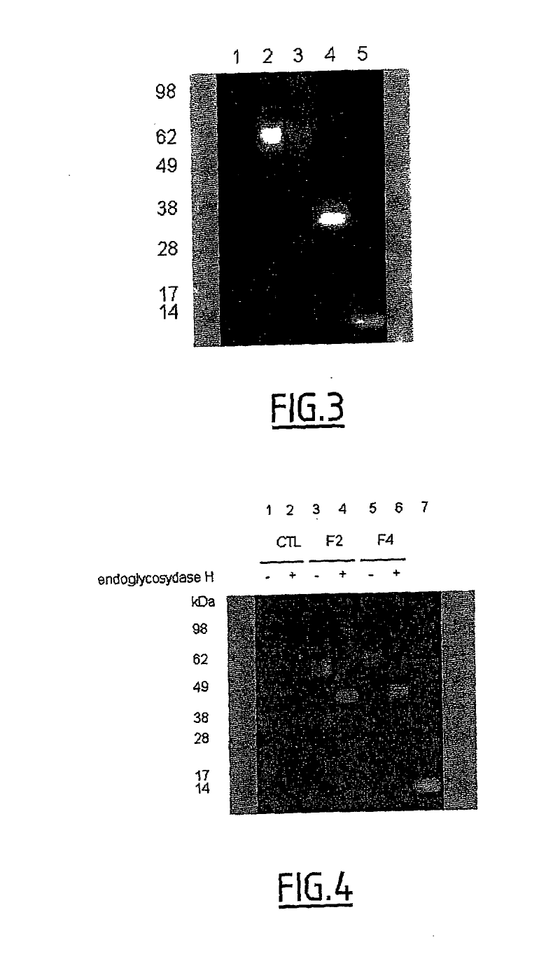

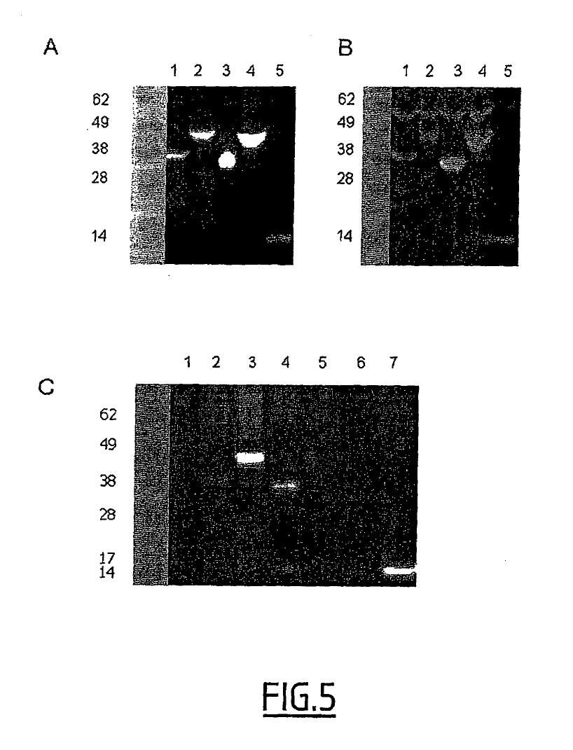

N-terminal and C-terminal fusions of Der p 2 with either mature Der p 1 or proDer p 1 were constructed. Boxes in FIG. 1 represent the different domains: Der p 2 (hatched), Der p 1 propeptide (white), mature Der p 1 (dotted). The numbers indicate the positions of the boundaries of these different moieties in the fusion proteins.
The four F1, F2, F3 and F4 fusion proteins have been expressed in E. coli. Those proteins are recognized by an anti-Der p 2 antibody and IgE from house dust mite allergic patients.
F2 and F4 fusion proteins have been successfully expressed in P. pastoris. These proteins are recognised by both anti Der p 1 and Der p 2 antibodies and by patients IgE. When expressed in yeast, F2 and F4 fusion proteins are hyperglycosylated. Their carbohydrates have been removed by enzymatic treatment with endoglycosidase H.

1. A fusion protein comprising a Group I allergen and a Group II allergen from the genus Dermatophagoides.

2. A fusion protein according to claim 1, wherein the Group II allergen is fused at the N- or C-terminal end of the Group I allergen.

3. A fusion protein according to claim 1, wherein said Group I allergen is Der p 1.

4. A fusion protein according to claim 1, wherein said Group I allergen is Der p 1 or proDer p 1, and said Group II allergen is Der p 2.

5. A fusion protein according to claim 1, wherein said Group I allergen is Der f 1 or proDer p 1 and said Group II allergen is Der f 2.

6. A fusion protein according to claim 1, comprising a sequence selected from the group consisting of SEQ ID NO:4, SEQ ID NO:5, SEQ ID NO:6 and SEQ ID NO: 7.

7. A fusion protein according to claim 1, comprising at least one additional allergen from the genus Dermatophagoides.

8. A fusion protein according to claim 1, comprising at least one addressing molecule targeting immune cells.

9. A nucleic acid comprising a sequence coding for a fusion protein comprising a Group I allergen and a Group II allergen from the genus Dermatophagoides.

10. A nucleic acid according to claim 9, which comprises a sequence consisting of a fusion of SEQ ID NO: 1 and SEQ ID NO: 2, or SEQ ID NO: 3 and SEQ ID NO: 2.

11. A nucleic acid according to claim 9, which comprises a sequence selected from the group consisting of SEQ ID NO:8, SEQ ID NO:9, SEQ ID NO:10 and SEQ ID NO:11.

12. An expression cassette comprising a sequence coding for a fusion protein comprising a Group I allergen and a Group II allergen from the genus ermatophagoides placed under the control of the elements necessary for its expression in a prokaryotic or eukaryotic cell or in an in vitro cell-free system.

13. An expression cassette according to claim 12, characterized in that the prokaryotic or eukaryotic cell is selected from cells of Escherichia coli, Pichia pastoris, Saccharomyces cerevisiae, COS and CHO cells.

14. A cloning and/or expression vector containing an expression cassette comprising a sequence coding for a fusion protein comprising a Group I allergen and a Group II allergen from the genus Dermatophagoides placed under the control of the elements necessary for its expression in a prokaryotic or eukaryotic cell or in an in vitro cell-free system.

15. A prokaryotic or eukaryotic cell containing an expression cassette according to claim 12.

16. A prokaryotic or eukaryotic cell according to claim 15 selected from the group consisting of a cell from Escherichia coli, Pichia pastoris or Saccharomyces cerevisiae, a COS cell, a CHO cell.

17. An immune or vaccinal composition comprising a fusion protein comprising a Group 1 allergen and a Group II allergen from the genus Dermatophagoides, in a pharmaceutically acceptable carrier.

18. The immune or vaccinal composition according to claim 17, wherein said fusion protein comprises a sequence selected from the group consisting of SEQ ID NO:4, SEQ ID NO:5, SEQ ID NO:6 and SEQ ID NO:7

19. An immune or vaccinal composition according to claim 17, further comprising a pharmaceutically acceptable adjuvant.

20. The immune or vaccinal composition according to claim 19, wherein the pharmaceutically acceptable adjuvant is a synthetic particulate vector that comprises a non-liquid hydrophilic core which comprises a cross-linked polysaccharide.

21. A method for preventing or treating an allergic reaction comprising administering an individual in need thereof with a prophylactically or therapeutically effective quantity of a fusion protein comprising a Group I allergen and a Group II allergen from the genus Dermatophagoides.

22. A method for in vitro detecting antibodies directed against a Group I allergen and/or Group II allergen, in a biological sample from an individual, which method comprises the steps consisting of:
a) contacting said biological sample from a patient with a fusion protein comprising a Group I allergen and a Group/I allergen from the genus Dermatophagoides;
b) detecting formation of immune complexes between said fusion protein and antibodies in the biological sample;
whereby, if immune complexes are detected, then the biological sample contains antibodies directed against a Group I allergen and/or a Group II allergen from the genus Dermatophagoides.

23. A method of detecting an IgE reactivity in an individual which comprises the steps of (a) injecting by intradermal or subcutaneous route a fusion protein comprising a Group I allergen and a Group II allergen from the genus Dermatophagoides; and (b) detecting a wheal and flare reaction at the site of injection, wherein a wheal and flare reaction is indicative of an IgE reactivity of the individual to a Group I and/or Group II allergen from the genus Dermatophagoides.

24. A diagnostic test for screening patients sensitized to Group I and/or Group II Dermatophagoides allergens, which comprises a fusion comprising a Group I allergen and a Group II allergen from the genus Dermatophagoides.
